(19) United States
(12) Patent Application Publication
(10) Pub. No.: US 2002/0094545 A1
(43) Pub. Date: Jul. 18, 2002

(54) Growth of human dendritic cells for cancer immunotherapy in closed system using microcarrier beads

(76) Inventors: Paul E. Harris, New York, NY (US); Charles Hesdorffer, Bronx, NY (US)

Correspondence Address: John P. White, Cooper & Dunham LLP, 1185 Avenue of the Americas, New York, NY, 10036, US

(21) Appl. No.: 09/726,883

(22) Filed: Nov. 30, 2000

(51) Int. Cl. G01N 33/567; C12N 5/08

(52) U.S. Cl. 435/7.21; 435/372

(57) ABSTRACT

A method of reproducibly generating dendritic cells is provided. Blood mononuclear cells is loaded into a cell culture container containing microcarrier beads therein. Tissue culture comprising the cells loaded in the container is incubated for a predetermined period. Nonadherent cells and cells adhered to the beads are separated. The method may further include preparing dendritic cell culture medium and transferring the dendritic cell culture medium to the container after the cells which are adhered to the beads are separated from the nonadherent cells. The tissue culture incubated for the predetermined time period may be washed to remove nonadherent cells. The beads may be allowed to settle and supernatant is expressed off. The container may comprise a gas permeable cell culture bag.

### BACKGROUND OF THE INVENTION

The present invention relates to a method of growing adherence-dependent hematopoietic cells. In particular, dendritic cells are grown in a closed system using microcarrier beads.

Dendritic cells (DCs) constitute potent antigen-presenting cells. They may be derived from bone marrow progenitor cells and circulate in small numbers in the peripheral blood. As antigen-presenting cells, DCs are able to induce activation of T-cells with a high degree of efficiency. They are highly specialized and optimally equipped for their task, since dendritic cells express molecules which are required for presenting antigen in large quantity. Important adhesion molecules, which guarantee intimate contact with the target cell, are present on the surface of the dendritic cells.

Due to low frequency of DC in peripheral blood, ex vivo expansion and maturation of DC precursors are required for their clinical application {J. Bartholeys et al., 149 Cellular Vaccines. Res. Immunol. 647-49 (1998)}.

There is a need to refine DC culture methods for clinical use in immunotherapy for cancer patients. Most DC culture systems are initiated from the adherent fraction of peripheral blood mononuclear cells, selected using open polystyrene flasks, followed by washing and then culture in serum-free medium containing GM-CSF and IL-4 or IL-7 (as well as other maturational cytokines) {G. Schuler et al., Dendritic cells as adjuvants for immune-mediated resistance to tumors, 186 J. Exp. Med. 1183-87 (1997); M. Di Nicola et al., Human dendritic cells: natural adjuvants in antitumor immunotherapy, 4 Cytokines Cell Mol. Ther. 265-73 (1998)). The open system is labor intensive and poses an increased risk of microbial contamination to the expanded product, the patient and the technician.

An alternative to the open flask is a closed system for culturing populations of monocyte enriched peripheral blood mononuclear cells using flexible gas permeable cell culture bags and sterile connecting devices {A. Glaser et al., Collection of mononuclear cells in the Spectra for the generation of dendritic cells. 39 Transfusion 661-62 (1999)}. Growing human DC in plastic bags, even under clinical grade and using good manufacturing practices, have poor yields because the surface of the bags is suboptimal.

### SUMMARY OF THE INVENTION

The present invention, in accordance with one embodiment, provides a method of reproducibly generating dendritic cells, comprising the steps of (a) loading blood mononuclear cells into a cell culture container containing microcarrier beads therein, (b) incubating for a predetermined time period tissue culture comprising the cells loaded in the container in step (a), and (c) separating nonadherent cells and cells adhered to the beads.

The present invention, in accordance with another embodiment, provides a method of reproducibly generating dendritic cells, comprising the steps of (a) loading microcarrier beads into a cell culture container, (b) loading blood mononuclear cells into the container, (c) incubating for a predetermined time period tissue culture comprising the mononuclear cells loaded in the container in step (b), and (d) separating nonadherent cells and cells adhered to the beads.

The container may comprise a gas permeable cell culture bag. The container is a closed vessel.

The tissue culture incubated for the predetermined time period may be washed to remove nonadherent cells. After the tissue culture is incubated for a predetermined time period, the beads may be allowed to settle and supernatant expressed off.

The method further may comprise (d) preparing dendritic cell culture medium, and (e) transferring the dendritic cell culture medium to the container after the nonadherent cells and the cells adhered to the beads are separated. The method further also may comprise (f) incubating the container for a second predetermined time period after step (e), (g) agitating contents of the container incubated in step (f), and (h) harvesting cell culture suspension by expression into transfer bags using a sterile connecting device after the beads agitated in step (g) are allowed to settle.

Samples may be removed from the container for quality control after the nonadherent cells and the cells adhered to the beads are separated. The quality control may include at least one of viability staining, microbial analysis, cell enumeration, microscopic examination of dendritic cell morphology, and immunophenotyping to determine a purity of the dendritic cell preparation.

The blood mononuclear cells may be obtained by apheresis.

### BRIEF DESCRIPTION OF THE DRAWINGS

The above and numerous features of the present invention would be more readily understood from the following detailed description by referring to the accompanying drawings wherein:

FIG. 1 provides a flow chart of a method of reproducibly generating dendritic cells, in accordance with an embodiment of the present invention;

FIG. 2 provides a flow chart of a method of reproducibly generating dendritic cells, in accordance with another embodiment of the present invention;

FIG. 3 provides a flow chart of a method, in accordance with another embodiment, for reproducibly generating dendritic cells;

FIG. 4 provides a table comparing dendritic cell culture methods;

FIG. 5 provides a table showing typical immunophenotype of cultured dendritic cells; and

FIGS. 6A through 6D provide a manufacturing flow sheet of a method, in accordance with another embodiment of the present invention, for reproducibly generating dendritic cells.

### DETAILED DESCRIPTION OF THE INVENTION

The present invention provides a novel and unobvious tool for reproducible generation of dendritic cells. Addition of selected, sterile plastic microcarrier beads enhances production of human dendritic cells (DC) in gas permeable cell culture bags. The method also may be adapted for growth of other adherence-dependent hematopoietic cells.

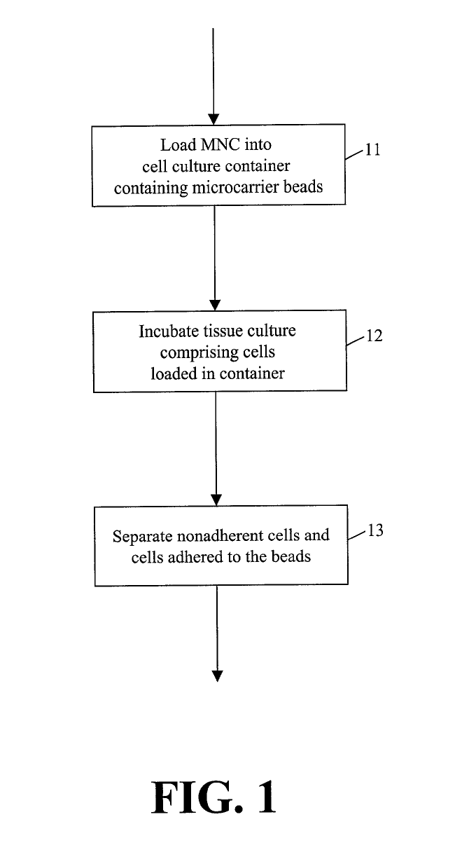

A method of reproducibly generating dendritic cells, in accordance with one embodiment of the present invention, will be described with reference to FIG. 1. Blood mononuclear cells are loaded into a cell culture container containing microcarrier beads (step 11). Tissue culture comprising the cells loaded in the container are incubated for a predetermined period (step 12). Nonadherent cells and cells adhered to the beads are separated (step 13).

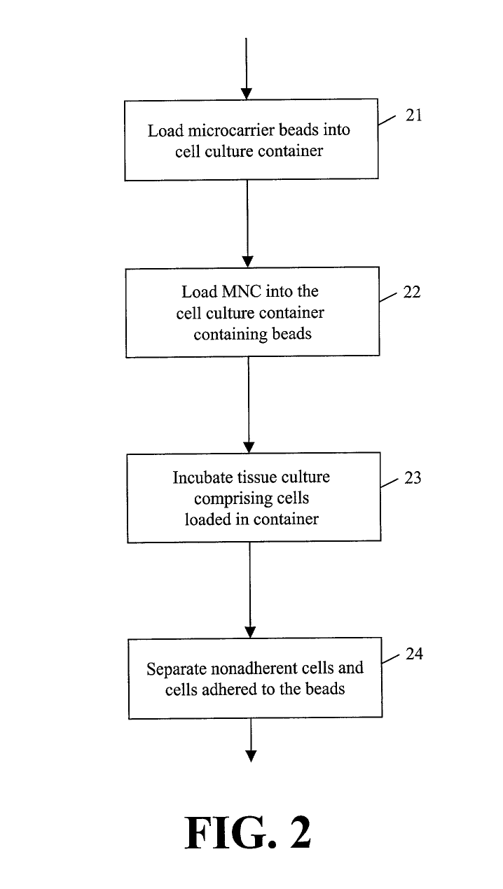

A method of reproducibly generating dendritic cells, in accordance with another embodiment of the present invention, includes first loading microcarrier beads into a cell culture container (step 21), as shown in FIG. 2. Blood mononuclear cells then are loaded into the container containing the microcarrier beads (step 22). Tissue culture comprising the cells loaded in the container are incubated for a predetermined period (step 23). Nonadherent cells and cells adhered to the beads are separated (step 24).

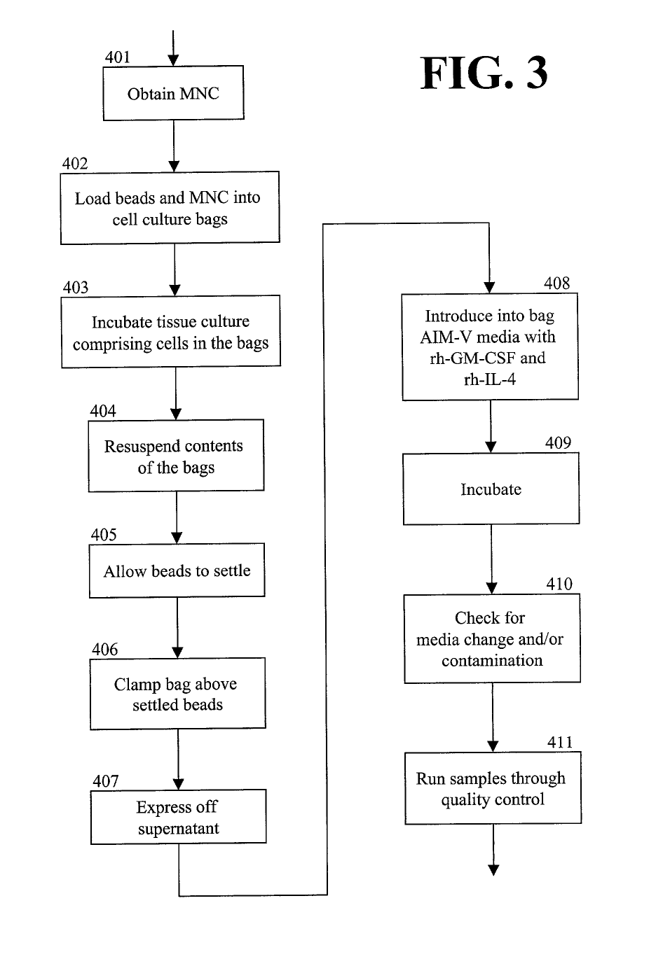

A more detailed description and additional embodiments are provided below with reference to FIGS. 3 through 6D.

One requirement of blood mononuclear cell (MNC) products suitable for DC culture is collection of a maximum number of monocytes and monocyte precursors with a minimum number of red blood cells, lymphocytes and platelets. This may be accomplished by pheresing donors on an apheresis system (e.g., Spectra, COBE BCT, Lakewood, Colo.) using a mononuclear cell program.

Thus, the MNC for the DC culture may be obtained (step 401) by apheresis, under informed consent, from G-CSF mobilized donors. Donors may undergo, for example, a 10-liter apheresis. The collection schema may utilize a separation fraction of 250, equivalent to a velocity of 635 rpm at an inlet flow of 50 ml per min. Materials and reagents used for the apheresis and DC culture preferably are sterile and/or endotoxin free and FDA approved for human use.

Without use of microcarrier beads, the yield of DCs per unit of culture surface area in closed gas permeable cell culture bags is less than the yield in open flask systems. To improve the yields of DCs in a closed system, styrene copolymer beads (e.g., 90-500 micron diameter, density ≦1.04 g/cm³, SoloHill Engineering, Inc., Ann Arbor, Mich.) are introduced into the bags, in accordance with one embodiment of the present invention, to increase the available surface area (e.g., by 380 cm²) and supply a surface area similar to that found in the flasks {see, e.g., M. Kiremitci et al., Cell adhesion to the surfaces of polymeric beads, 18 Biomater. Artif. Cells Artif. Organs 599-603 (1990)}. Also, the beads have a density that allows them to sink/settle in due course.

In accordance with one (FIGS. 3 through 5) of many possible embodiments, 1 gram of gamma radiation sterilized beads and 10×10*8 total cells/bag of MNC product are diluted in 100 mls AIM-V (e.g., from GIBCO, Grand Island, N.Y.) and loaded into gas permeable cell culture bags (e.g., Lifecell X-fold Cell Culture Containers PL2417, 180 cm², Nexell Therapeutics, Irvine, Calif.), under a biological safety cabinet (step 402).

The tissue culture bags then are incubated (step 403), for example, in a humidified 37° C., 5% CO₂ atmosphere for approximately four hours. After four hours the contents of the bag are gently resuspended (step 404), the beads are allowed to settle for 5 minutes at 1× g (step 405), and the bag is clamped 1 cm above the settled beads (step 406). The supernatant then is expressed off (step 407) using a transfer bag and a sterile connecting device (e.g, from Terumo Corp., Phoenix, Ariz.). This procedure (steps 402-407) may be repeated three times with 50 mls AIM-V media. As control for adherence, a sample of the expressed cells may be immunophenotyped for monocyte markers (e.g., CD14 and CD11c). Adherence of MNC to the bag and bead surface may be inferred by a decrease in the percent of CD14 and CD11c positive cells in the expressed fraction relative to the apheresis product.

After removal of nonadherent cells, 100 ml of AIM-V media containing rh-GM-CSF (e.g., 25 ng/ml, Sargramostim, Immunex, Seattle, Wash.) and rh IL-4 (e.g., 1000U/ml, Sigma, St. Louis, Mo.) is introduced into the tissue culture bags (step 408). The bags may be placed into a dedicated, Hepa-filtered, humidified 37° C. 5% CO₂ incubator (step 409) for 7 days. At day 3 or 4, the bags are visually inspected to check for media color change or bacterial/fungal contamination (step 410). On day 7 (although the culture period may be as little as four days), the tissue culture bags may be removed from the incubator and samples removed therefrom for quality control, e.g., viability staining, microbial analyses, cell enumeration using a hematology analyzer (e.g., from Beckman-Coulter, Hialeah, Fla.), microscopic examination of dendritic cell morphology, and immunophenotyping to determine the purity of the dendritic cell preparation (step 411). Immunophenotyping may be performed using a flow cytometer (e.g., FACSCalibur, Becton Dickinson, San Jose, Calif.) and corresponding software (e.g., CellQuest, Becton Dickinson, San Jose, Calif.). The monoclonal antibody panel may include antibodies to CD45/CD14, CD3/CD19, CD1a, CD11c, HLA-DR, CD83, CD86 and CD123.

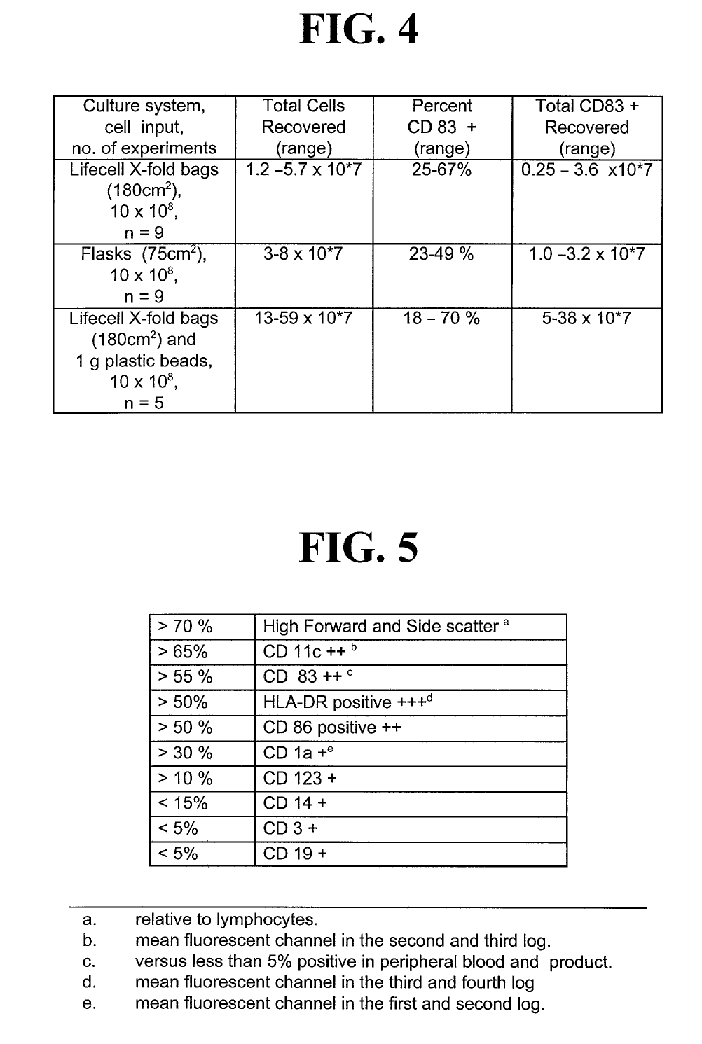

In experiments using the method described above, plastic beads were not visible in the supernatant on microscopic examination. The yields of DC are improved when compared to the other systems studied (see FIG. 4). The immunophenotype of the recovered cells (see FIG. 5) meets established DC phenotypes effective in adjuvant vaccine therapy. Culture supernatants are routinely negative for microbial contamination.

The quantities of the cells produced are acceptable for adaptive transfer strategies. Current tumor antigen vaccine protocols require approximately 10⁷ to 10⁸ total DCs. Using this closed system of culture, a sufficient number of DCs can be harvested for a complete course of therapy using a single 10-liter MNC apheresis and an average of five culture bags.

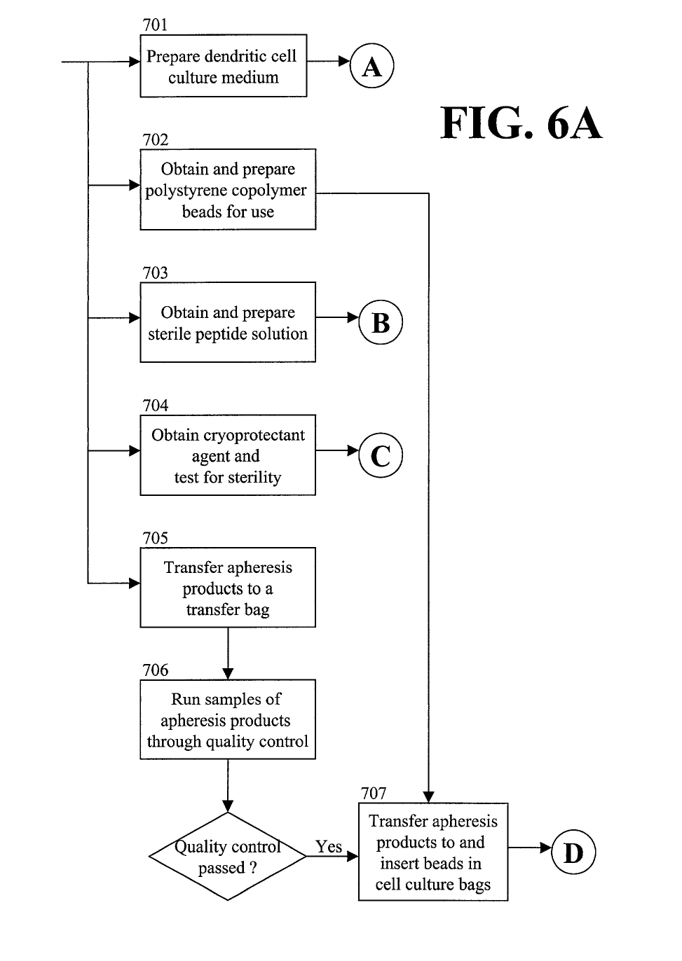

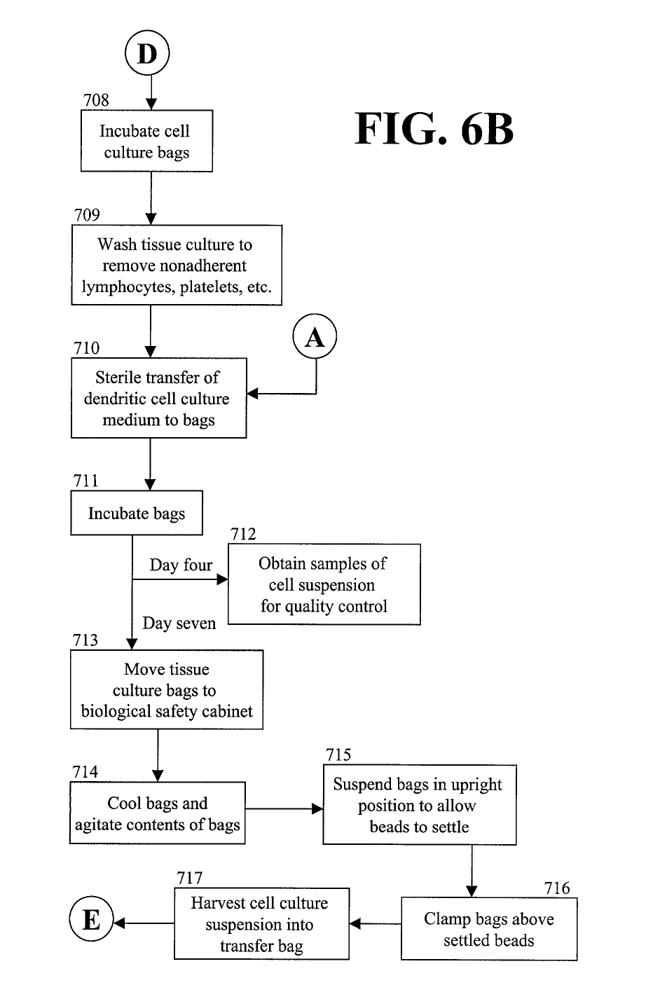

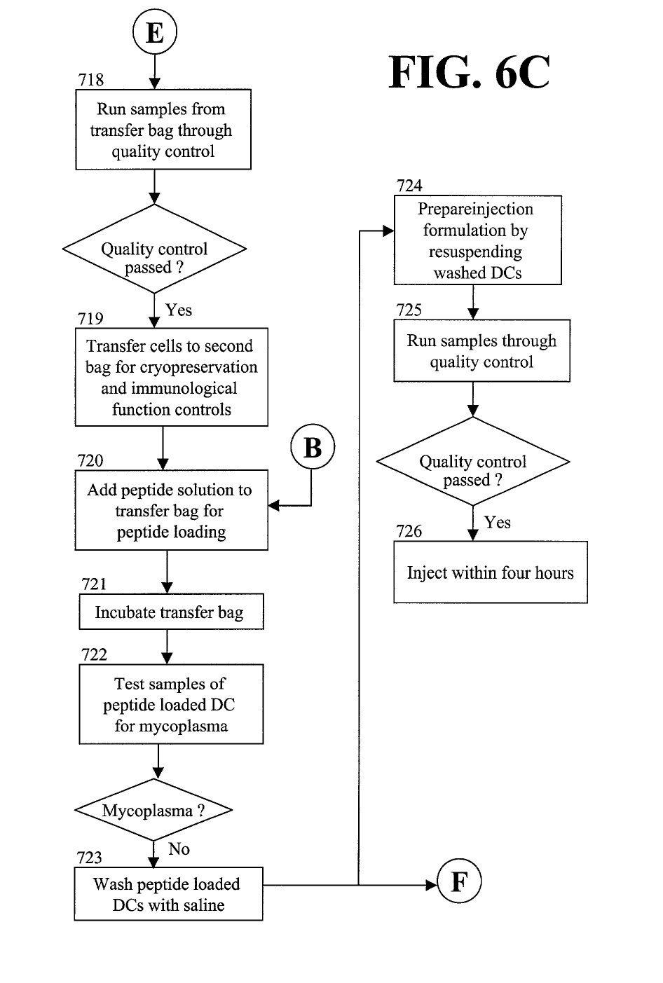

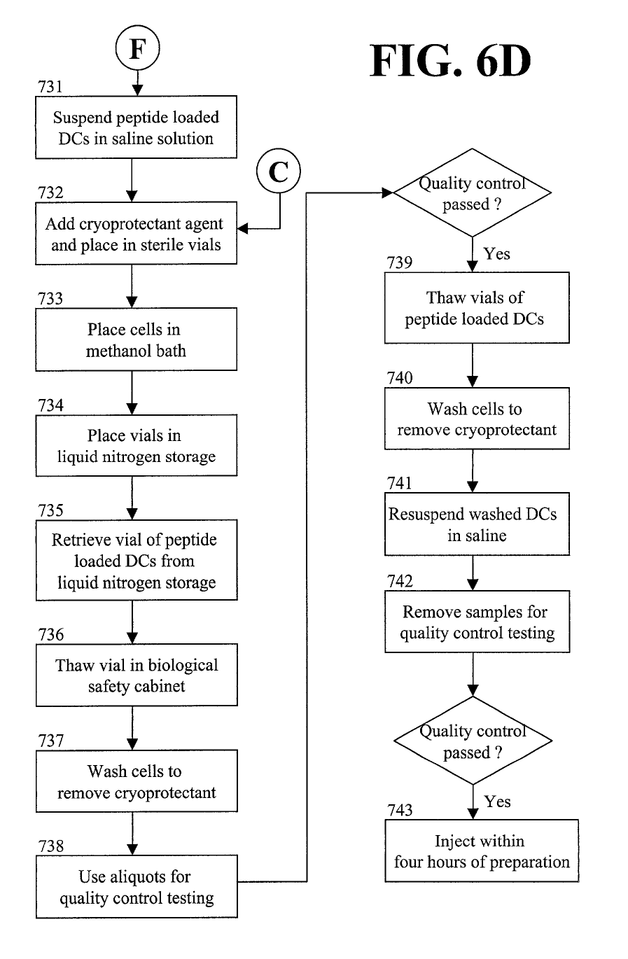

Another embodiment will now be described with reference to FIGS. 6A through 6D.

A dendritic cell culture medium is prepared (step 701) by combining AIM V media (e.g., BB-MF 2557, Life Technologies, Grand Island, N.Y.), rh-IL-4 (e.g., 1000 U/ml, GLP grade, Sigma Aldrich, St. Louis, Mo.) and rh-GM-CSF (e.g., 25 ng/ml, Therapeutic grade, Immunex, Seattle, Wash.).

Polystyrene copolymer beads (e.g., 250 micron diameter, density=1.07 g/cm³, BB-MF 3094, Solohill Engineering, Inc., Ann Arbor, Mich.) are obtained and prepared (step 702) for use. For example, the beads may be suspended in a phosphate buffered saline (e.g., EDR9865, therapeutic grade, Nexell, Calif.) [100 gms beads/200 mls saline], placed in an autoclavable glass bottle and capped, and sterilized in an autoclave (e.g., 20 lbs/sq.in. at 121° C. for 1 hour with slow exhaust). The container is sealed and then transferred to a biological safety cabinet. A 1 ml aliquot is removed, placed in the transport tube, and tested for sterility (e.g., Bioscreen Testing Services, Inc., Torrance, Calif.). The polystyrene beads are used if no bacterial growth is detected.

Also, a sterile peptide (e.g., HER-2, or another peptide antigen specific to another target tumor) solution is prepared (step 703), using for example synthetic peptide (e.g., GLP grade, American Peptide Company) and phosphate buffered saline. For example, HER-2 synthetic peptide powder is dissolved in saline at a concentration of 200 ug/ml (20×) and sterile filtered through 0.2 micron nylon membrane. The solution is aliquoted in sterile 10 ml vials and stored.

A cryoprotectant agent (e.g., DMSO, USP grade, Gaylord Chemical Corporation, Slidell, La.) is obtained and tested for sterility (step 704).

Apheresis products are transferred to a transfer bag (step 705). Samples of the apheresis products are run through quality control (e.g., hematology analyzer, Trypan blue viability, CD45/14 immunophenotype)[step 706]. If quality control is passed, apheresis products (e.g., 10×10⁸ mononuclear cells/bag×four to five bags) are transferred (step 707) from the transfer bag using a sterile connecting device (e.g., Lifecell transfer set, Nexell Therapeutics, Irvine, Calif.) to gas permeable tissue culture bags (e.g., therapeutic grade, Lifecell X-fold Cell Culture Containers PL2417, 180 cm², Nexell). The beads that pass quality control (step 702) also are inserted in the bags.

The tissue culture bags then are incubated (step 708), for example, in a humidified 37° C., 5% CO₂ atmosphere for approximately four hours. At the midpoint, the bag is flipped from one side to the other. After four hours, the tissue culture is washed three times with AIM-V to remove nonadherent lymphocytes, platelets, grans, RBC, etc. (step 709). The wash includes transfer of the AIM-V media and expressing off the supernatant while leaving the beads in the bag.

Next, the dendritic cell culture medium (prepared in step 701) is transferred via a sterile process to the tissue culture bags (step 710). The bags are incubated again, for example, in a humidified dedicated 37° C. 5% CO₂ incubator (step 711) for 5 to 7 days. At day 4, samples of cell suspension are removed for quality control (step 712). On day 7, the tissue culture bags are moved from the incubator to a biological safety cabinet (step 713). The bags are cooled to room temperature, and the contents are gently agitated for five minutes (step 714). The bags are suspended in an upright position to allow the beads to settle for 5 minutes at 1× g (step 715), and the bag is clamped above the settled beads (step 716). The cell culture suspension is harvested (step 717) by expression into transfer bags (e.g., Stericell bags, Nexell Therapeutics) using a sterile connecting device (e.g, from Terumo Corp., Phoenix, Ariz.).

Samples are removed from the transfer bags and run through quality control (step 718). For example, if no beads are present and viability is greater than 95%, then the samples are passed to immunophenotype by flow cytometry. If quality control is passed, cells (e.g., approximately 50×10⁶ cells or any range, such as all of the cells) can be transferred (step 719) to a second bag for cryopreservation and immunological function controls (e.g., seven day proliferation assay using harvested cells as stimulators for lymphocytes from three different individuals).

HER-2 peptide solution is added to the transfer bag (step 720) for peptide loading onto the HLA Class I of the DC (final concentration is 10 ug/ml). The transfer bag is incubated (step 721) overnight (e.g., 4 to 12 hours at 37° C., 5% CO₂ in a humidified atmosphere of a dedicated incubator). Samples of the peptide loaded DC are tested for mycoplasma (step 722). If the test results are negative for mycoplasma, the peptide loaded DCs are washed three times with therapeutic grade phosphate buffer saline (step 723). In a preclinical phase, samples of the suspension may be removed for quality control analysis, such as for endotoxin (e.g., USP LAL), fluoride (ion specific electrode) and residual organic solvent (GC-MS).

Injection formulation is prepared by resuspending the washed DCs (step 724) at a concentration less than 10⁷ cells/ml (e.g., 3, 6, 9 or 12×10⁶ cells/ml) in saline supplemented with 5% autologous serum obtained the same day. Samples are removed for quality control (step 725), such as Gram stain. If quality control is passed, the injection formulation is cleared for administration and injected within four hours of preparation (step 726).

The remaining cells are cryopreserved. First, the peptide loaded dendritic cells (also DC without peptide) are suspended (step 731) in a solution of therapeutic grade saline supplemented with 5% autologous serum (5×10⁶/ml). Cryoprotectant agent is added to a final concentration of 10% and placed in sterile NUNC vials (5 ml) [step 732]. The cells are placed in a methanol bath at −70° C. overnight (step 733), then placed in vapor phase liquid N₂ storage until use (step 734).

After two to three days of storage, a vial of peptide loaded DC is retrieved (step 735) from the liquid N₂ storage for quality control testing. Vials are thawed at 37° C. in a biological safety cabinet (step 736). The cells are washed with AIM-V to remove cryoprotectant (step 737).

Aliquots are removed for the following assays:

| | |
|---|---|
| Viability | (>70%) |
| Sterility USP | (No growth) |
| Mycoplasma by PCR | (negative) |
| MLC test (7 day) | (Stimulate proliferative response |
| | greater that 3 × BACKGROUND at |
| | responder to stimulator ratio of |
| | 10 to 1) |
| Endotoxin (USP LAL) | <0.06 EU/ml |

If the quality control test (step 738) is passed, the cryopreserved DC are released for thawing. Vials of cryopreserved peptide loaded DC are thawed (step 739) at 37° C. in the biological safety cabinet. Cells are washed with therapeutic grade saline three times to remove cryoprotectant agent (step 740). Washed DC are resuspended (step 741) in saline supplemented with 5% autologous serum obtained the same day. Samples are removed for quality control testing (step 742), e.g., viability staining and Gram stain. If viability is greater than 70%, then the cells are passed to adjust cell concentration to less than 10⁷ viable cells/ml (e.g., 6, 9 or 12×10⁶ viable cells/ml) ×1 ml. Next, a Gram stain is applied. If the Gram stain is passed, the injection formulation is cleared for administration and injected within four hours of preparation (step 743).

The use of polystyrene beads in closed containers has a number of advantages over the use of open flasks for reproducibly generating dendritic cells, including sterility, risks of exposure for workers, higher yield, etc. Because of these factors, a closed system, e.g., flexible gas permeable plastic tissue culture bags, is preferred over the open flasks. The bags alone, however, do not provide an ideal surface for the attachment of DC precursor cells (monocytes). The introduction of selected polystyrene beads into the bags provides a surface that the monocytes easily adhere to. Once the monocytes have matured into DC, their adherence to the polystyrene surface, provided by the beads, is significantly reduced. At the end of the culture period, DCs no longer adhere to the beads and are harvested in the supernatant.

The beads are selected based, in part, on their size. Since more surface area is desirable, smaller beads in a larger quantity is preferred to larger beads in a smaller quantity.

Also, the specific gravity of the beads which allows them to settle after a period also contributes to their utility in the above-described methods. Since the monocytes adhered to the beads settle with the beads and thereby separate from the undesired cells (e.g., lymphocytes, platelets, etc.) which are removed by expressing off the supernatant.

Quality control is applied in the methods described above to comply with good manufacturing practices criteria.

While the invention has been described with reference to specific embodiments, the description is not meant to be construed in a limiting sense. It should be understood that the invention is not limited to the precise embodiments described herein.

For example, while the closed system in the embodiments described above use a cell culture bag, other cell culture vessels may be used for the closed container. The container need not have any particular shape. It is preferable, but not essential, to have more than one port on the container to facilitate the transfer of materials in and out of the container. It is important, however, that the container is gas permeable to, for example, O₂ and CO₂. Also, it is important to maintain a ratio of (beads and container) surface area to container volume that allows the container to hold enough media to support the culture period, so that culture only needs to be fed once, rather than repeatedly.

Improvements and modifications which become apparent to persons of ordinary skill in the art after reading this disclosure, the drawings and the appended claims are deemed within the spirit and scope of the present invention. It is therefore contemplated that the appended claims would cover any such modifications or improvements.

What is claimed is:

1. A method of reproducibly generating dendritic cells, comprising the steps of:
(a) loading blood mononuclear cells into a cell culture container containing microcarrier beads therein;
(b) incubating for a predetermined time period tissue culture comprising the cells loaded in the container in step (a); and
(c) separating nonadherent cells and cells adhered to the beads.

2. A method of reproducibly generating dendritic cells, comprising the steps of:
(a) loading microcarrier beads into a cell culture container;
(b) loading blood mononuclear cells into the container;
(c) incubating for a predetermined time period tissue culture comprising the mononuclear cells loaded in the container in step (b); and
(d) separating nonadherent cells and cells adhered to the beads.

3. The method of claim 1, wherein the container comprises a gas permeable cell culture bag.

4. The method of claim 1, wherein the container is a closed vessel.

5. The method of claim 1, wherein the tissue culture incubated for the predetermined time period in step (b) is washed to remove nonadherent cells.

6. The method of claim 1, wherein after step (b) the beads are allowed to settle and supernatant is expressed off.

7. The method of claim 1 further comprising:
(d) preparing dendritic cell culture medium; and
(e) transferring the dendritic cell culture medium prepared in step (d) to the container after step (c).

8. The method of claim 7 further comprising:
(f) incubating the container for a second predetermined time period after step (e);
(g) agitating contents of the container incubated in step (f); and
(h) harvesting cell culture suspension by expression into transfer bags using a sterile connecting device after the beads agitated in step (g) are allowed to settle.

9. The method of claim 1, wherein after step (c) samples are removed from the container for quality control.

10. The method of claim 9, wherein the quality control includes at least one of viability staining, microbial analysis, cell enumeration, microscopic examination of dendritic cell morphology, and immunophenotyping to determine a purity of the dendritic cell preparation.

11. The method of claim 1, wherein the blood mononuclear cells are obtained by apheresis.

12. The method of claim 1, wherein a ratio of a combined surface area of the microcarrier beads and the container to a volume of the container volume is a value that allows the container to hold enough media for the predetermined time period of incubation in step (b).
